(19) United States
(12) Patent Application Publication
(10) Pub. No.: US 2022/0112536 A1
(43) Pub. Date: Apr. 14, 2022

(54) OXYGEN-INSENSITIVE ELECTROCHEMICAL BIOSENSOR AND METHODS OF USE THEREOF

(71) Applicant: Trividia Health, Inc., Fort Lauderdale, FL (US)

(72) Inventors: Ngoc Minh Phuong Bui, Coconut Creek, FL (US); Janette Lackore, Weston, FL (US); Jessica Gendron, Margate, FL (US); Aaron Ivey, Fort Lauderdale, FL (US)

(21) Appl. No.: 17/497,422

(22) Filed: Oct. 8, 2021

Related U.S. Application Data
(60) Provisional application No. 63/089,260, filed on Oct. 8, 2020.

(51) Int. Cl. C12Q 1/00; C12N 9/04; G01N 33/543; G01N 33/66; C12Q 1/32; G01N 27/327

(52) U.S. Cl. CPC C12Q 1/006; C12N 9/0006; G01N 33/54388; G01N 27/3273; C12Q 1/32; G01N 27/3272; G01N 33/66

(57) ABSTRACT

The present disclosure provides a reagent for detecting an analyte, comprising: an enzyme comprising glucose oxidoreductase; at least one electroactive molecule selected from resazurin, resazurin salt, resorufin or resorufin salt; and at least one metal coordination complex.

### RELATED APPLICATIONS

This application claims priority to and the benefit of U.S. Provisional Patent Application No. 63/089,260, filed Oct. 8, 2020, the contents of which is incorporated herein in its entirety.

### FIELD

The present disclosure relates to electrochemical sensors and, more particularly, to systems and methods for electrochemically sensing blood glucose levels.

### BACKGROUND

Many industries have a commercial need to monitor the concentration of particular constituents in a fluid. For example, patients suffering from diabetes must carefully monitor their blood glucose levels on a daily basis. A number of systems that allow people to conveniently monitor their blood glucose levels are available. Such systems typically include a test strip where the user applies a blood sample and a meter that “reads” the test strip to determine the glucose level in the blood. Among the various technologies available for measuring blood glucose levels, electrochemical technologies are particularly desirable because only a very small blood sample may be needed to carry out the measurement. One feature of some conventional miniaturized electrochemical test strips is the presence of a single layer of biological reagents over both the working and counter electrodes. However, due to the biological nature of the reagent layer, it is difficult to reproducibly manufacture each strip with the exact same sensitivity. The best reagent compositions are those that exhibit increased sensitivity by facilitating the free flow of electrons between the sample being analyzed and the electrode and its connected circuitry. Accurate measurement of blood glucose levels may be critical to the long-term health of many users. As a result, meters and test strips used to measure blood glucose levels should be highly reliable. Certain meter and test strip compositions have shown sensitivity to oxygen, thus affecting overall performance. Accordingly, there exists a need for improved biological reagent compositions with reduced sensitivity to oxygen in measuring systems and methods.

### SUMMARY

As described below, the present disclosure features compositions and methods for electrochemically sensing blood glucose levels.

In some aspects, the present disclosure provides a reagent for detecting an analyte, comprising: an enzyme comprising glucose oxidoreductase; at least one electroactive molecule selected from resazurin, resazurin salt, resorufin or resorufin salt; and at least one metal coordination complex.

In some embodiments, the glucose oxidoreductase is glucose dehydrogenase. In some embodiments, the at least one metal coordination complex comprises a ruthenium complex or an osmium complex. In some embodiments, the at least one electroactive molecule is provided in concentration of between 0.01 mM and about 20 mM. In some embodiments, the at least one electroactive molecule comprises resazurin or resazurin salt is provided in concentration of between 1 mM and about 3 mM. In some embodiments, the at least one electroactive molecule comprises resorufin or resorufin salt is provided in concentration of between 1 mM and about 4 mM. In some embodiments, the reagent can further comprise a coenzyme comprising flavin adenine dinucleotide or nicotinamide adenine dinucleotide.

In some aspects, the present disclosure provides a test strip for detecting an analyte, comprising: a substrate with a proximal region and a distal region; a conductive pattern formed on the substrate including at least one electrode disposed on the substrate at the proximal region of the strip, electrical strip contacts disposed on the substrate at a conductive region at the distal region of the test strip, and conductive traces electrically connecting the electrodes to at least some of the electrical strip contacts; and a reagent layer contacting at least a portion of at least one electrode, the reagent layer comprising: a glucose oxidoreductase; a mediator formulation comprising at least one electroactive molecule independently selected from resazurin, resazurin salt, resorufin or resorufin salt; and at least one metal coordination complex.

In some embodiments, the glucose oxidoreductase is glucose dehydrogenase. In some embodiments, the at least one metal coordination complex comprises a ruthenium complex or an osmium complex. In some embodiments, the at least one electroactive molecule is provided in concentration of between 0.01 mM and about 20 mM. In some embodiments, the at least one electroactive molecule comprises resazurin or resazurin salt is provided in concentration of between 1 mM and about 3 mM. In some embodiments, the at least one electroactive molecule comprises resorufin or resorufin salt is provided in concentration of between 1 mM and about 4 mM. In some embodiments, the reagent layer can further include a coenzyme comprising flavin adenine dinucleotide or nicotinamide adenine dinucleotide.

In some aspects, the present disclosure provides a system for measuring a body fluid constituent comprising: a test strip for detecting an analyte comprising: a substrate with a proximal region and a distal region; a conductive pattern formed on the substrate including at least one electrode disposed on the substrate at the proximal region of the strip, electrical strip contacts disposed on the substrate at a conductive region at the distal region of the test strip, and conductive traces electrically connecting the electrodes to at least some of the electrical strip contacts; and a reagent layer contacting at least a portion of at least one electrode, the reagent layer comprising: a glucose oxidoreductase; a mediator formulation comprising at least one electroactive molecule independently selected from resazurin, resazurin salt, resorufin or resorufin salt; and at least one metal coordination complex; and a diagnostic meter configured to receive the test strip and to electrically connect to the electrical strip contacts.

In some embodiments, the glucose oxidoreductase is glucose dehydrogenase. In some embodiments, the at least one metal coordination complex comprising a ruthenium complex or an osmium complex. In some embodiments, the at least one electroactive molecule is provided in a concentration of between 0.01 mM and about 20 mM. In some embodiments, the at least one electroactive molecule comprises resazurin or resazurin salt and is provided in a concentration of between 1 mM and about 3 mM. In some embodiments, the at least one electroactive molecule comprises resorufin or resorufin salt and is provided in a concentration of between 1 mM and about 4 mM.

### BRIEF DESCRIPTION OF THE DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1A is a general cross-sectional view of a test strip according to some embodiments of the present disclosure.

FIG. 1B is a top view of a conductive pattern on a substrate of a test strip according to some embodiments of the present disclosure.

FIGS. 2A and 2B illustrate a meter according to some embodiments of the present disclosure.

FIG. 3 is a schematic of electron transfer flow and measured current as a function of glucose concentration for sensor strips having different mediator formulations.

FIG. 4 is a graph of percent bias from non-oxygenated blood versus partial pressure of oxygen (pO₂) level in mmHg comparing Meldola's blue, Resazurin and Resorufin.

FIG. 5 is a graph of percent bias from non-oxygenated blood versus partial pressure of oxygen (pO₂) at various pH levels for 25 mg/dL glucose, comparing pH 6.5 (squares), pH 7.2 (circles), and pH 7.7 (triangles).

FIG. 6 is a graph of percent bias from non-oxygenated blood versus partial pressure of oxygen (pO₂) for various concentrations of resazurin sodium salt in 20 mg/dL glucose, comparing 1× (1.2 mM) resazurin sodium salt (squares), 2× (2.4 mM) resazurin sodium salt (triangles), and 3× (3.6 mM) resazurin sodium salt (circles).

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the presently disclosed embodiments

Subject matter will now be described more fully with reference to the accompanying drawings, which form a part hereof, and which show, by way of illustration, specific example aspects and embodiments of the present disclosure. Subject matter may, however, be embodied in a variety of different forms and, therefore, covered or claimed subject matter is intended to be construed as not being limited to any example embodiments set forth herein; example embodiments are provided merely to be illustrative. The following detailed description is, therefore, not intended to be taken in a limiting sense.

Among the various technologies available for measuring blood glucose or other analyte levels, electrochemical technologies are particularly desirable because only a very small blood sample may be needed to carry out the measurement. One feature of some conventional miniaturized electrochemical test strips is the presence of a single layer of biological reagents over both the working and counter electrodes. However, due to the biological nature of the reagent layer, it is difficult to reproducibly manufacture each strip with the exact same sensitivity. The best reagent compositions are those that exhibit increased sensitivity by facilitating the free flow of electrons between the sample being analyzed and the electrode and its connected circuitry.

Certain meter and test strip compositions have shown sensitivity to oxygen, thus affecting overall performance. This disclosure relates to compositions of electrochemical biosensors for measuring analytes of interest (e.g., glucose). Biosensors using ruthenium hexaammine chloride as the primary electron transfer medium have advantages in manufacturing and storage due to their higher redox stability with small change in the background current signal, when coupled with a secondary mediator, for example, 8-dimethylamino-2,3-benzophenoxazine hemi(zinc chloride) salt (Meldola' s Blue). However, these biosensors can still be affected by the oxygen partial pressure. Therefore, there is a need to develop formulations that are not influenced by oxygen, that have robust performance at different temperature and humidity, are stable for long term storage, and are suitable for mass production.

In this disclosure, the reagent composition comprises an enzyme that facilitates the oxidation or reduction of glucose, for example, oxidoreductase (e.g., glucose dehydrogenase, glucose oxidase),transferase (e.g. hexokinase). In some embodiments, the enzyme can be used with or without a cofactor (e.g., flavin, nicotinamide, quinone derivatives), a ruthenium-based redox mediator (e.g., hexaammine ruthenium, biphosphine ruthenium), and a secondary mediator comprising at least one electroactive molecule (e.g., resazurin, resazurin salt, resorufin or resorufin salt) with a reduced sensitivity to oxygen in blood. In some embodiments, the secondary mediator may be a chemical that displays an improvement in sensitivity to oxygen. In some embodiments, the at least one electroactive molecule is resazurin sodium salt or resorufin sodium salt.

In particular, the at least one electroactive molecule of the secondary mediator can be selected such that percent bias between the glucose result of the oxygenated samples, in which the partial pressure of oxygen concentration is ≥75 mmHg, and non-oxygenated samples, in which the partial pressure of oxygen concentration is <75 mmHg is between −1% and −10%. In some embodiments, the percent bias may be between −1% and −8%. In some embodiments, the percent bias may be between −1% and −6%. In some embodiments, the at least one electroactive molecule is independently selected from resazurin, resazurin salt, resorufin or resorufin salt and results in the percent bias between −1% and −10%. In some embodiments, the at least one electroactive molecule is independently selected from resazurin, resazurin salt, resorufin or resorufin salt and results in the percent bias between −1% and −8%. In some embodiments, the at least one electroactive molecule is independently selected from resazurin, resazurin salt, resorufin or resorufin salt and results in the percent bias between −1% and −6%. In some embodiments, the at least one electroactive molecule of the secondary mediator is selected such that test strips containing Meldola's Blue are between 2 and 11 times more sensitive to oxygen than the test strip of the present disclosure. In some embodiments, the at least one electroactive molecule is selected from resazurin, resazurin salt, resorufin or resorufin salt such that test strips containing Meldola's Blue are between 2 and 11 times more sensitive to oxygen than the test strip with resazurin, resazurin salt, resorufin or resorufin salt.

In some embodiments, the systems and methods of the present disclosure can be used to determine glucose concentration in a blood sample for monitoring a diabetic patient. In some embodiments, the glucose concentration can be used to determine the amount of lactose in milk or other dairy products. For example, the amount of glucose formed during the hydrolysis of lactose is proportional to the amount of lactose present in the sample. Accordingly, the glucose concentration in the dairy sample can be used to calculate the amount of lactose in the sample. In some embodiments, the present composition may be used in many other diagnostic applications testing for glucose or any other analyte.

System Overview

A meter for measuring blood glucose can include a portable, handheld device used to measure blood glucose levels for users with Type I or Type II diabetes. Typically, the user purchases test strips that interface with the meter. The user draws a tiny amount of blood (a few microliters or less) from a finger or other area using a lancet and a blood droplet is applied onto the exposed end of the strip which has an open port for the blood. The strip is inserted into the meter connector port and a chemical and/or electrochemical reaction occurs between the blood sample and the reagent composition on the strip, which is measured by the meter to determine the blood glucose level in units of mg/dL or mmol/L, depending on regional preferences.

In a typical electrochemical system, the oxidation or reduction half-cell reaction involving glucose either produces or consumes electrons. This electron flow can be measured, provided the electrons can interact with a working electrode that is in contact with the sample to be analyzed. The electrical circuit is completed through a counter electrode that is also in contact with the sample. A chemical reaction occurs at the counter electrode, and this reaction (oxidation or reduction) is the opposite of the reaction at the working electrode. See, for example, Fundamentals Of Analytical Chemistry, 4th Edition, D. A. Skoog and D. M. West; Philadelphia: Saunders College Publishing (1982), pp 304-341.

Test Strip

The test strip includes a reagent layer comprising at least one of the reagent compositions described herein. An individual test strip may also include an embedded code relating to data associated with a lot of test strips, or data particular to that individual strip. Such coded strips are further described in U.S. Pat. Pub. No. 2007/0015286, which is herein incorporated by reference in its entirety. The test strip may include a sample chamber for receiving a user's fluid sample, such as, for example, a blood sample. The sample chamber and test strip can be formed using materials and methods described in U.S. Pat. No. 6,743,635, which is herein incorporated by reference in its entirety. Accordingly, the sample chamber may include a first opening in the proximal end of the test strip and a second opening for venting the sample chamber. The sample chamber may be dimensioned so as to be able to draw the blood sample in through the first opening, and to hold the blood sample in the sample chamber, by capillary action. The test strip can include a tapered section that is narrowest at the proximal end, or can include other indicia in order to make it easier for the user to locate the first opening and apply the blood sample.

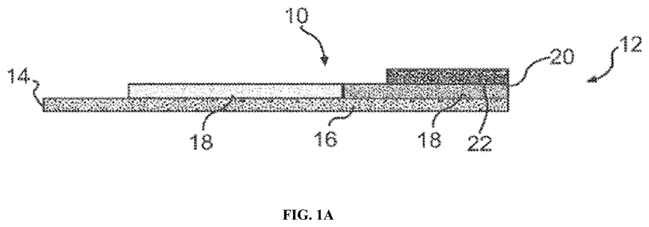

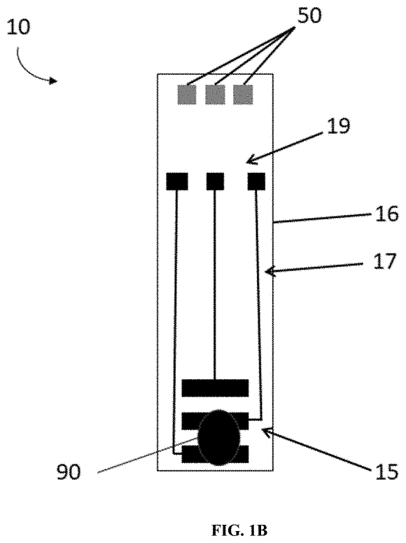

FIG. 1A illustrates a general cross-sectional view of an example embodiment of a test strip 10. In particular, FIG. 1A depicts a test strip 10 that includes a proximal end 12, a distal end 14, and is formed with a base layer 16 extending along the entire length of test strip 10. The base layer 16 can be composed of an electrically insulating material and has a thickness sufficient to provide structural support to test strip 10. For purposes of this disclosure, “distal” refers to the portion of a test strip further from the fluid source (e.g., closer to the meter) during normal use, and “proximal” refers to the portion closer to the fluid source (e.g., a fingertip with a drop of blood for a glucose test strip) during normal use. The base layer 16 may be composed of an electrically insulating material and has a thickness sufficient to provide structural support to test strip 10.

As seen in FIG. 1A, the proximal end 12 of test strip 10 includes a sample receiving location, such as a sample chamber 20 configured to receive a patient's fluid sample, as described above. The sample chamber 20 may be formed in part through a slot in a dielectric insulating layer 18 formed between a cover 22 and the underlying measuring electrodes formed on the base layer 16. Accordingly, the sample chamber 20 may include a first opening, e.g., a sample receiving location, in the proximal end of the test strip and a second opening for venting the sample chamber 20. The sample chamber 20 may be dimensioned to be able to draw the blood sample in through the first opening, and to hold the blood sample in the sample chamber 20, by capillary action. The test strip 10 can include a tapered section that is narrowest at the proximal end 12, or can include other indicia to make it easier for the user to locate the first opening and apply the blood sample.

In reference to FIG. 1B, in accordance with an example embodiment of the present disclosure, the strip 10 can include a conductive pattern disposed on base layer 16 of the strip 10. In some embodiments, the conductive pattern may be formed by laser ablating the electrically insulating material of the base layer 16 to expose the electrically conductive material underneath. Other methods may also be used, such as inserted conductors with physical attachment to control circuit. Other methods may also be used to dispose the conductive pattern on the base layer. The conductive pattern may include a plurality of electrodes 15 disposed on base layer 16 near proximal end 12, a plurality of electrical strip contacts 19 disposed on base layer 16 near distal end 14, and a plurality of conductive traces 17 electrically connecting the electrodes 15 to the plurality of electrical strip contacts 19.

In some embodiments, a calibration code can be included on the test strip. In some embodiments, the calibration code can be included on the test strip in the form of a second plurality of electrical strip contacts near the distal end of the strip. The second plurality of electrical contacts can be arranged such that they provide, when the strip is inserted into the meter, a distinctly discernable calibration code specific to the test strip lot readable by the meter. As noted above, the readable code can be read as a signal to access data, such as calibration coefficients, from an on-board memory unit in the meter related to test strips from that lot, or even information corresponding to individual test strips. For example, as shown in FIG. 1B, the test strip can include a calibration code in the form of a plurality of contacts 50.

In some embodiments, a reagent layer 90, or reagent composition, may be disposed on the base layer 16 of the strip 10 in contact with at least a working electrode of the conductive pattern as shown in FIG. 1B and described further below.

Blood Glucose Meter

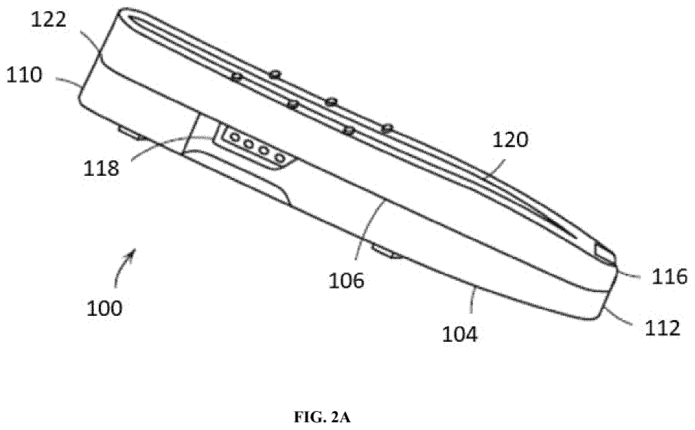

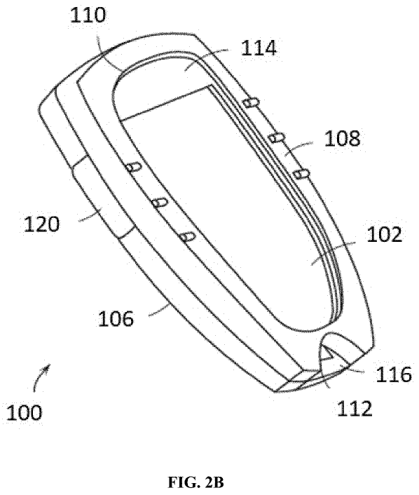

FIG. 2A and FIG. 2B illustrate an exemplary illustration of a meter 100 used to measure the glucose level in a blood sample. The meter 100 includes a housing having a test port for receiving the test strip, and a processor or microprocessor programmed to perform methods and algorithms to determine glucose concentration in a test sample or control solution as disclosed in the present disclosure. In some embodiments, the meter 100 has a size and shape to allow it to be conveniently held in a user's hand while the user is performing the glucose measurement. The meter 100 may include a front side 102, a back side 104, a left side 106, a right side 108, a top side 110, and a bottom side 112. The front side 102 may include a display 114, such as a liquid crystal display (LCD). A bottom side 112 may include a strip connector 116 into which test strip can be inserted to conduct a measurement. The meter 100 may also include a storage device for storing test algorithms or test data. The left side 106 of the meter 100 may include a data connector 418 into which a removable data storage device 120 may be inserted, as necessary. The top side 110 may include one or more user controls 122, such as buttons, with which the user may control meter 100, and the right side 108 may include a serial data connector (not shown).

The meter may be battery powered and may stay in a low-power sleep mode when not in use in order to save power. When the test strip is inserted into the meter, the first and second plurality of electrical contacts on the test strip contact corresponding electrical contacts in the meter. The second plurality of electrical contacts may bridge a pair of electrical contacts in the meter, causing a current to flow through a portion of the second plurality of electrical contacts. The current flow through the second plurality of electrical contacts causes the meter to wake up and enter an active mode. The meter also reads the code information provided by the second plurality and can then identify, for example, the particular test to be performed, or a confirmation of proper operating status. In addition, the meter can also identify the inserted strip as either a test strip or a check strip based on the particular code information. If the meter detects a check strip, it performs a check strip sequence. If the meter detects a test strip, it performs a test strip sequence.

In the test strip sequence, the meter validates the working electrode, counter electrode, and, if included, the fill-detect electrodes, by confirming that there are no low-impedance paths between any of these electrodes. If the electrodes are valid, the meter indicates to the user that the sample may be applied to the test strip. The meter then applies a drop-detect voltage between the working and counter electrodes and detects a fluid sample, for example, a blood sample, by detecting a current flow between the working and counter electrodes (i.e., a current flow through the blood sample as it bridges the working and counter electrodes). To detect that an adequate sample is present in the sample chamber and that the blood sample has traversed the reagent layer and mixed with the chemical and biological constituents in the reagent layer, the meter may apply a fill-detect voltage between the fill-detect electrodes and measures any resulting current flowing between the fill-detect electrodes. If this resulting current reaches a sufficient level within a predetermined period of time, the meter indicates to the user that adequate sample is present and has mixed with the reagent layer.

The meter can be programmed to wait for a predetermined period of time after initially detecting the blood sample, to allow the blood sample to react with the reagent layer or can immediately begin taking readings in sequence. During a fluid measurement period, the meter applies an assay voltage between the working and counter electrodes and takes one or more measurements of the resulting current flowing between the working and counter electrodes. The assay voltage is near the redox potential of the formulation in the reagent layer, and the resulting current is related to the concentration of the particular constituent measured, such as, for example, the glucose level in a blood sample. In some embodiments, the working and counter electrodes are produced on a conductive substance.

Reagent Composition

In some embodiments, a reagent composition, or reagent layer, is provided for use in an electrochemical sensor for measuring the glucose level in a sample. The reagent composition includes an oxidoreductase, a mediator formulation, and optionally, a coenzyme. The mediator formulation comprises at least one electroactive molecule and at least one coordination complex. Reagent layers of the present disclosure may provide for improvements in accuracy, sensitivity, range of analysis, and stability.

In some embodiments, a reagent layer 90, or reagent composition, may be disposed on the base layer 16 of the strip 10 in contact with at least a working electrode of the conductive pattern (FIG. 1B). In some embodiments, the reagent layer features a single layer of biological reagents over both the working and counter electrodes. The use of a single reagent layer can provide for simple manufacturing of the strips, since only one deposition step is needed to coat the material onto the electrodes. The reagent layer may include an enzyme that facilitates the oxidation-reduction reaction of glucose, such as a glucose oxidoreductase, optionally a coenzyme, and a mediator or mediators, such as potassium ferricyanide or ruthenium hexaammine, that help to transfer electrons between the oxidation-reduction reaction and the working electrode. Reagent layer 90 may also include other components, such as buffering materials (e.g., potassium phosphate), polymeric binders (e.g., hydroxypropyl-methyl-cellulose, sodium alginate, microcrystalline cellulose, polyethylene oxide, hydroxyethylcellulose, and/or polyvinyl alcohol), and surfactants (e.g., Triton X-100 or Surfynol 485). With these chemical constituents, the reagent layer reacts with glucose in the blood sample in the following way. The glucose oxidoreductase initiates a reaction that initiates the redox cascade. In some embodiments, the glucose oxidoreductase oxidizes the glucose to gluconic acid and reduces the ferricyanide to ferrocyanide. When an appropriate voltage is applied to the working electrode, relative to the counter electrode, the ferrocyanide is oxidized to ferricyanide, thereby generating a current that is related to the glucose concentration in the blood sample. As would be appreciated by one skilled in the art, any combination of strips 10 known in the art can be utilized without departing from the scope of the present disclosure.

A “sample” may include a composition containing an unknown amount of the analyte (e.g., glucose) of interest. Typically, a sample for electrochemical analysis is in liquid form, and preferably the sample is an aqueous mixture. A sample may be a biological sample, such as blood, urine or saliva. A sample may be a derivative of a biological sample, such as an extract, a dilution, a filtrate, or a reconstituted precipitate. A sample may be a sample derived from milk and other dairy products. A sample may be a sample derived from grapes and wine and other winery products.

An “oxidoreductase” may include any enzyme that facilitates the oxidation or reduction of a substrate. The oxidoreductase may include one or more of “oxidases,” which facilitate oxidation reactions in which molecular oxygen is the electron acceptor; “reductases,” which facilitate reduction reactions in which the analyte is reduced, and molecular oxygen is not the analyte; or “dehydrogenases,” which facilitate oxidation reactions in which molecular oxygen is not the electron acceptor. See, for example, Oxford Dictionary of Biochemistry and Molecular Biology, Revised Edition, A. D. Smith, Ed., New York: Oxford University Press (1997) pp. 161, 476, 477, and 560, which is herein incorporated by reference in its entirety. Examples of oxidoreductases includes glucose dehydrogenase (GDH), glucose oxidasecholesterol esterase, lipoprotein lipase, glycerol kinase, lactate oxidase, pyruvate oxidase, alcohol oxidase, uricase, and the like. In some embodiments, the oxidoreductase may be a glucose oxidoreductase, that is, an oxidoreductase enzyme that facilitates the oxidation or reduction of glucose. In some embodiments of the present disclosure the oxidoreductase is GDH.

A “coenzyme” may include a non-protein redox prosthetic. Coenzymes of the present disclosure are preferably organic molecules that are linked covalently or noncovalently to an enzyme and are changed, for example, oxidized or reduced, by the conversion of the analyte. Examples of coenzymes are flavin, nicotinamide and quinone derivatives, for example: flavin nucleotide derivatives such as FAD, FADH₂, FMN, FMNH₂, etc.; nicotinamide nucleotide derivatives such as NAD⁺, NADH, NADP⁺, NADPH; or ubiquinones such as coenzyme Q or PQQ. In some embodiments of the present disclosure the coenzyme is a flavin nucleotide.

A “mediator” may include a substance that can be oxidized or reduced and that can transfer one or more electrons between a first substance and a second substance. A mediator is a reagent in an electrochemical analysis and is not the analyte of interest. In a simple system, the mediator undergoes a redox reaction with the oxidoreductase after the oxidoreductase has been reduced or oxidized through its contact with an appropriate substrate. This oxidized or reduced mediator then undergoes the opposite reaction at the electrode and is regenerated to its original oxidation number.

A “coordination complex” may include a complex having well-defined coordination geometry, such as octahedral or square planar geometry. Unlike organotransition metal complexes, which are defined as complexes where a transition metal is bonded to at least one carbon atom through a sigma bond, coordination complexes are defined by their geometry. Thus, coordination complexes may be organotransition metal complexes (such as ferricyanide (III) and its reduced ferrocyanide (II) counterpart), or complexes where non-metal atoms other than carbon, such as heteroatoms including nitrogen, sulfur, oxygen, and phosphorous, are datively bonded to the transition metal center. For example, ruthenium hexaammine is a coordination complex having a well-defined octahedral geometry where six NH₃ ligands (formal charge of 0 on each of the 6 ligands) are datively bonded to the ruthenium center. A more complete discussion of organotransition metal complexes, coordination complexes, and transition metal bonding may be found in Collman et al., Principles and Applications of Organotransition Metal Chemistry (1987) and Miessler & Tarr, Inorganic Chemistry (1991), which are herein incorporated by reference in their entireties. In some embodiments of the present disclosure the coordination complex is selected from iron, ruthenium and osmium complexes. In some embodiments, the coordination complex is ruthenium hexaammine.

An “electroactive molecule” may include an organic molecule that does not contain a metal and that is capable of undergoing an oxidation or reduction reaction. Electroactive molecules can behave as redox species and as mediators. Examples of electroactive molecules include benzoquinones and naphthoquinones, N-oxides, nitroso compounds, hydroxylamines, oximes, phenazines, phenothiazines, phenoxazines, indophenols, and indamines. Other examples of electroactive molecules include those described in U.S. Pat. No. 5,520,786, which is herein incorporated by reference in its entirety. Other examples of electroactive molecules include those described in U.S. Pat. No. 8,062,490, which is herein incorporated by reference in its entirety. In some embodiments, the electroactive molecule may be in a salt form (e.g., sodium, potassium, calcium, magnesium, etc.). In some embodiments, the electroactive molecule may be in an alkali metal salt form (e.g., lithium, sodium, potassium, rubidium, cesium, etc.).

In some embodiments of the present disclosure, at least one electroactive molecule is independently selected from phenazines, phenothiazines, or phenoxazines, or salts thereof (e.g., phenoxazinium salts). Phenoxazines and their salts include, but are not limited to, benzophenoxazines and their corresponding salts (i.e., benzophenoxazinium salts). In an embodiment, the at least one electroactive molecule is a phenoxazine salt.

In some embodiments of the present disclosure, at least one electroactive molecule is independently selected from 7-Hydroxy-3H-phenoxazin-3-one 10-oxide (resazurin) or a resazurin salt. In some embodiments, at least one electroactive molecule is independently selected from 7-Hydroxy-3H-pherioxazin-3-one (resorufin) or a resorufin salt. In some embodiments, the resazurin salt or resorufin salt can be a salt of sodium, potassium, calcium, magnesium, or a similar metal. In some embodiments, the resazurin salt or resorufin salt can be in an alkali metal salt form (e.g., lithium, sodium, potassium, rubidium, cesium, or similar). In some embodiments, the resazurin salt is 7-Hydroxy-3H-phenoxazin-3-one-10-oxide sodium salt (resazurin sodium salt). In some embodiments, the resorufin salt is 7-Hydroxy-3H-phenoxazin-3-one sodium salt (resorufin sodium salt).

In an embodiment, glucose dehydrogenase (GDH) and flavin adenine dinucleotide (FAD) comprise the oxidoreductase-coenzyme combination. In another embodiment, GDH and nicotinamide adenine dinucleotide (NAD+) comprise the oxidoreductase-coenzyme combination. In another embodiment, the oxidoreductase is glucose oxidase. In addition, a mediator formulation comprising an electroactive molecule and a coordination complex may be incorporated into the reagent layer with the oxidoreductase-coenzyme. In an embodiment, a phenoxazine salt (e.g., resazurin sodium salt) and a ruthenium complex (e.g., ruthenium hexaammine) are combined with GDH-FAD. In another embodiment, a phenoxazine salt (e.g., resazurin sodium salt) and a ruthenium complex (e.g., ruthenium hexaammine) are combined with GDH-NAD+.

During a sample test, the GDH initiates a reaction that oxidizes the glucose to gluconic acid and reduces the coordination complex (e.g., resazurin to resorufin and ruthenium (III) to ruthenium (II)). When an appropriate voltage is applied to a working electrode, relative to a counter electrode, the reduced coordination complex (e.g., ruthenium (II)) is oxidized (e.g., ruthenium (III)), thereby generating a current that is related to the glucose concentration in the blood sample. The meter then calculates the glucose level based on the measured current and on calibration data that the meter has been signaled to access by the code data read from the second plurality of electrical contacts associated with the test strip. The meter then displays the calculated glucose level to the user.

In some embodiments, the reagent composition of the present disclosure remains insensitive to oxygen over a wide concentration of the at least one electroactive molecule in the secondary mediator (e.g., independently selected from resazurin, resazurin salt, resorufin or resorufin salts) such as, for example, in concentration of between about 0.01 mM and about 20 mM. In some embodiments, the at least one electroactive molecule may be provided in concentration of between about 1 mM and about 3 mM. In some embodiments, the at least one electroactive molecule may be provided in concentration of between about 1 mM and about 2 mM. In some embodiments, the at least one electroactive molecule is resazurin or resazurin salt provided in concentration between about 0.01 mM and about 20 mM. In some embodiments, the at least one electroactive molecule is resazurin or resazurin salt provided in concentration between about 1 mM and about 3 mM. In some embodiments, the at least one electroactive molecule is resazurin or resazurin salt provided in concentration between about 1 mM and about 2 mM. In some embodiments, the at least one electroactive molecule is resorufin or resorufin salt provided in concentration between about 0.01 mM and about 4 mM. In some embodiments, the at least one electroactive molecule is resorufin or resorufin salt provided in concentration between about 1 mM and about 4 mM. In some embodiments, the at least one electroactive molecule is resorufin or resorufin salt provided in concentration between about 2 mM and about 3 mM.

In some embodiments, the enzyme may be provided in concentration of between about 0.5 g/L and about 50 g/L. In some embodiments, the enzyme may be provided in concentration of between about 5 g/L and about 10 g/L. In some embodiments, the enzyme (e.g., glucose oxidase) may be provided in concentration of between about 0.5 g/L and about 25 g/L. In some embodiments, the enzyme (e.g., glucose oxidase) may be provided in concentration of between about 5 g/L and about 10 g/L. In some embodiments, the oxidoreductase-coenzyme combination (e.g., GDH-FAD) may be provided in concentration of between about 100 U/mL and about 10,000 U/mL. In some embodiments, the oxidoreductase-coenzyme combination may be provided in concentration of between about 1000 U/mL and about 5000 U/mL. In some embodiments, the oxidoreductase-coenzyme combination may be provided in concentration of between about 1000 U/mL and about 2500 U/mL. In some embodiments, the oxidoreductase-coenzyme combination may be provided in concentration of between about 2500 U/mL and about 5000 U/mL. In some embodiments, the ruthenium complex may be provided in concentration of between about 50 mM and about 500 mM. In some embodiments, the ruthenium complex may be provided in concentration of between about 100 mM and about 300 mM. In some embodiments, the ruthenium complex may be provided in concentration of between about 130 mM and about 170 mM. In some embodiments, the ruthenium complex (e.g., hexaammine ruthenium chloride) may be provided in concentration of between about 50 mM and about 175 mM.

Optionally, the reagent composition may include inert ingredients that are not directly involved in any oxidation-reduction reactions in the electrochemical sensor. Examples of such inert ingredients include binding agents, thickening agents, and buffering components. Binding agents may include bentone, polyethylene oxide, carboxymethyl cellulose, polyvinyl alcohol, polyacrylic acid, methocel, CMC, PEG and/or PEO. Thickening agents may include silica and/or polyethylene oxide. Buffering components may be made up of one or more, e.g., two, three, four or more, distinct buffering agents, where the buffering component stabilizes the mediator during storage of the composition in dry form such that little if any of the mediator is reduced prior to use, e.g., during storage. A buffer is considered to stabilize a mediator if, in the presence of the buffer, little if any of the mediator converts to a reduced form over a given storage period. Suitable buffers are buffers that do not cause the background signal in an electrochemical test to increase over time. Example buffers include, but are not limited to, succinate, TES, BES, ACES, HEPES, MOPS, phosphate, bis-tris propane, and/or imidazole. The background signal is the signal obtained when analyte-free sample is introduced to the electrochemical testing system.

In addition, the reagent composition may further include a wetting agent. In some embodiments, the wetting agent is used in combination with a detergent. Wetting agents may be added to facilitate uniform coating of the reagent composition onto an electrochemical test strip. A plurality of one or more of the combination of agents may also be used. The agents used may improve dissolution of the assay reagents as well as enhance the wicking properties of a capillary fill strip. The agents include those known in the art, for example, polymers, anti-foaming agents, and surfactants. Representative types of surfactants/detergents of interest include, but are not limited to: Tritons, Macols, Tetronics, Silwets, Zonyls, and Pluronics. Suitable agents include Pluronic materials which are block co-polymers of polyethylene oxide and polypropylene oxide. Examples of Pluronic materials include Pluronic P103 which has good wetting properties and Pluronic F87 Prill which has good detergent properties. Both Pluronic P103 and F87 Prill also have a cloud point temperature greater than 80° C. which is desirable since this property avoids a phase change in the composition during the drying process.

Some reagent compositions may also include one or more enzyme cofactors. Enzyme cofactors of interest include divalent metal cations such as Ca²⁺ and/or Mg²⁺.

Stabilizers may also be added to the reagent composition to help stabilize the enzyme and prevent denaturation of the protein. The stabilizer may also help stabilize the redox state of the mediator, in particular, the oxidized redox mediator. Examples of stabilizing agents include, but are not limited to carbohydrates (e.g. sucrose, trehalose, mannitol, and lactose), amino acids, proteins (e.g. BSA and albumin) and organic compounds such as EDTA and the like, as well as gluconate, calcium, magnesium, and/or glutamate.

Viscosity modifiers may also be added to the reagent to modify the liquid reagent rheology. Examples of such agents include poly(acrylic acid), poly(vinyl alcohol), dextran, and/or BSA.

In some embodiments, the reagent layer may react with glucose in the blood sample in order to determine the particular glucose concentration.

The following examples provide additional embodiments of the disclosure.

### EXAMPLE 1

### Comparison of Oxygen Sensitivity Between Meldola's Blue, Resazurin Sodium Salt, and Resorufin Sodium Salt

Glucose test strips were fabricated with Meldola's Blue, or resazurin sodium salt, or resorufin sodium salt in the mediator formulation and tested via amperometry. Blood samples at various partial pressure of oxygen was applied to the test strip. A non-oxygenated blood sample was used as the control. The concentration of glucose within each sample was the same. The partial pressure of oxygen and glucose concentration of each aliquot of blood was determined by a blood gas analyzer and glucose analyzer, respectively. The percent bias between the glucose result of the oxygenated and non-oxygenated sample was calculated.

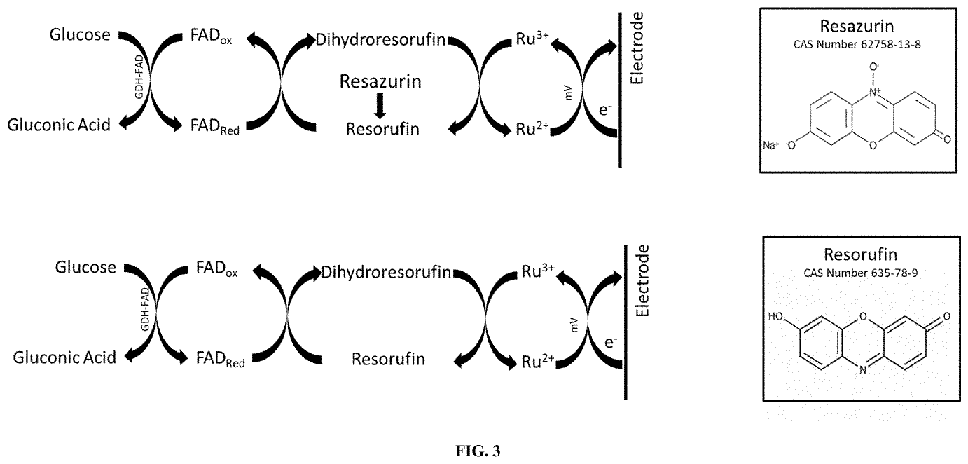

FIG. 3 is a schematic of electron transfer flow and measured current as a function of glucose concentration for sensor strips having different mediator formulations, namely, resazurin and resorufin.

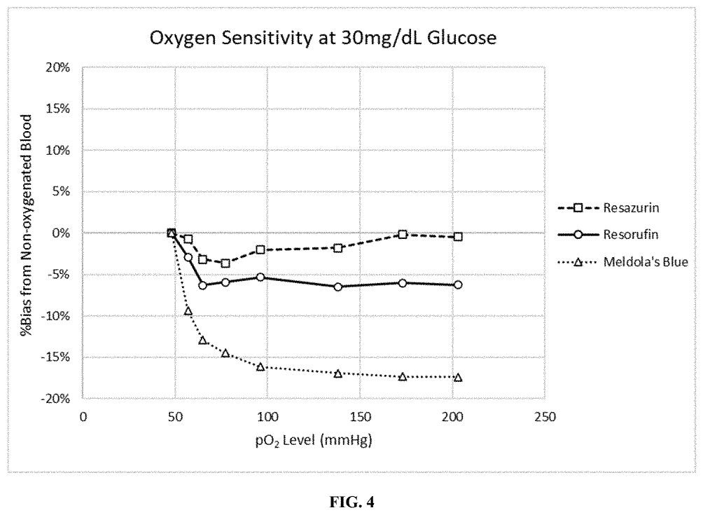

FIG. 4 is a graph of percent bias from non-oxygenated blood versus partial pressure of oxygen (pO₂) level in mmHg comparing Meldola's blue, resazurin sodium salt, and resorufin sodium salt, showing that resazurin sodium salt and resorufin sodium salt is less sensitive to oxygen than Meldola's Blue. In particular, on average, the bias between the glucose result of the oxygenated samples, in which the partial pressure of oxygen concentration is ≥75 mmHg, and non-oxygenated samples, in which the partial pressure of oxygen concentration is <75 mmHg, is −16% for test strips containing Meldola's blue; whereas the bias is −1.6% and −6.0% for test strips containing Resazurin sodium salt and Resorufin sodium salt, respectively. Therefore, test strips containing Resazurin sodium salt or Resorufin sodium salt are 10.2 or 2.7 times less sensitive to oxygen than test strips containing Meldola' s Blue, respectively.

### EXAMPLE 2

### Oxygen Effect of Resazurin Sodium Salt at Neutral pH at 25 mg/dL Glucose

Glucose test strips were fabricated with 1.2 mM resazurin sodium salt at pH 6.5, 7.2, and 7.7 in the mediator formulation and tested via amperometry. Blood samples at various partial pressure of oxygen was applied to the test strip. A non-oxygenated blood sample was used as the control. The concentration of glucose within each sample was the same. The partial pressure of oxygen and glucose concentration of each aliquot of blood was determined by a blood gas analyzer and glucose analyzer, respectively. The percent bias between the glucose result of the oxygenated and non-oxygenated sample was calculated.

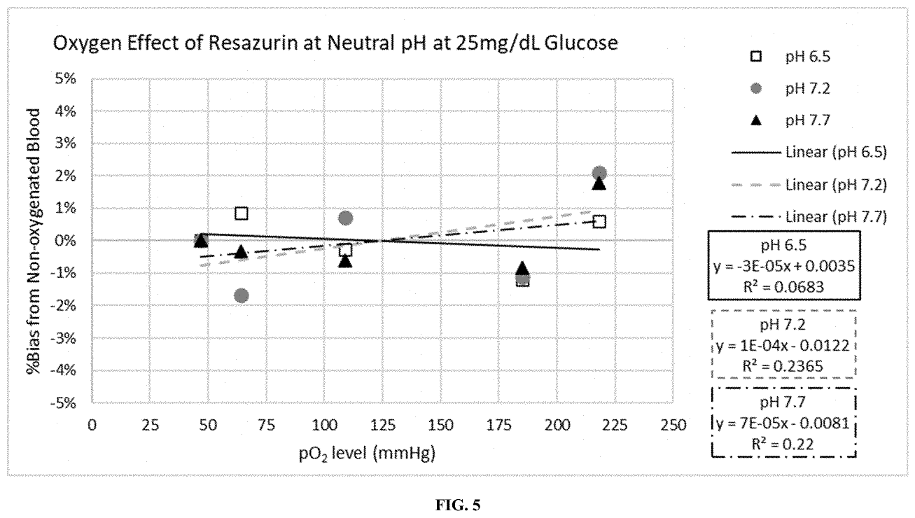

FIG. 5 is a graph of percent bias from non-oxygenated blood versus partial pressure of oxygen (pO₂) at various pH levels for 25 mg/dL glucose, comparing pH 6.5 (squares), pH 7.2 (circles), and pH 7.7 (triangles), showing that resazurin sodium salt is insensitive to oxygen at neutral pH range.

### EXAMPLE 3

### Oxygen Effect at Various Concentrations of Resazurin Sodium Salt at 20 mg/dL Glucose

Glucose test strips were fabricated with 1.2 mM, 2.4 mM, and 3.6 mM resazurin sodium salt in the mediator formulation and tested via amperometry. Blood samples at various partial pressure of oxygen was applied to the test strip. A non-oxygenated blood sample was used as the control. The concentration of glucose within each sample was the same. The partial pressure of oxygen and glucose concentration of each aliquot of blood was determined by a blood gas analyzer and glucose analyzer, respectively. The percent bias between the glucose result of the oxygenated and non-oxygenated sample was calculated.

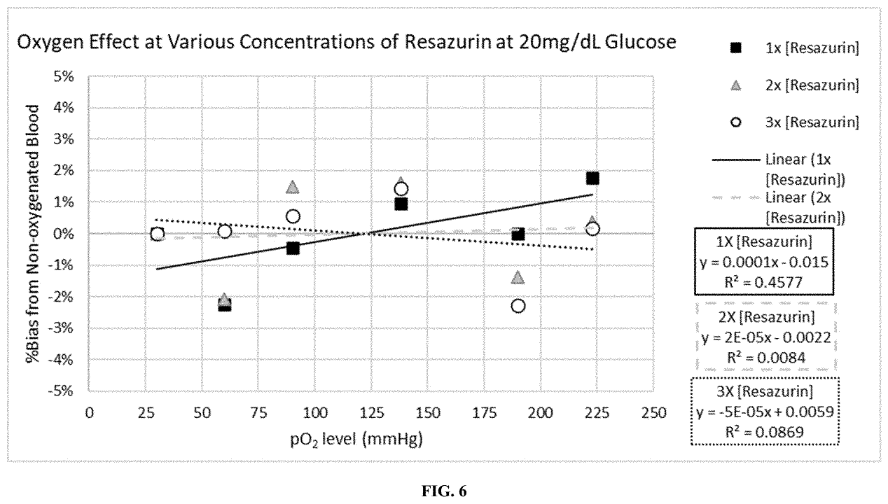

FIG. 6 is a graph of percent bias from non-oxygenated blood versus partial pressure of oxygen (pO₂) for various concentrations of resazurin sodium salt in 20 mg/dL glucose, comparing 1× (1.2 mM) resazurin sodium salt (squares), 2× (2.4 mM) resazurin sodium salt (triangles), and 3× (3.6 mM) resazurin sodium salt (circles), showing that the chemical reagent layer of the disposable test strip remains insensitive to oxygen over a wide concentration of resazurin sodium salt.

Exemplary Embodiments of the Disclosure

In some embodiments, the reagent layer may react with glucose in the blood sample in order to determine the particular glucose concentration. The reagent layer comprises an oxidoreductase, a coenzyme, and a mediator formulation. The mediator formulation comprises at least one electroactive molecule and at least one coordination complex. The at least one electroactive molecule may be independently selected from phenoxazines, or salts thereof. The at least one coordination complex may be independently selected from iron, osmium or ruthenium complexes. In an embodiment, the at least one electroactive molecule is a phenoxazine or a salt thereof salt and the at least one coordination complex is a ruthenium complex.

In some aspects, the present disclosure provides a reagent for detecting an analyte, comprising a glucose oxidoreductase; a mediator formulation comprising at least one electroactive molecule independently selected from resazurin, resazurin salt, resorufin or resorufin salt; and at least one metal coordination complex. In some embodiments, the glucose oxidoreductase is glucose dehydrogenase. In some aspects, the present disclosure further involves a coenzyme. In some embodiments, the coenzyme is flavin adenine dinucleotide. In some embodiments, the coenzyme is nicotinamide adenine dinucleotide. In some embodiments, the at least one metal coordination complex is a ruthenium complex or an osmium complex.

In some aspects, the present disclosure provides a test strip for detecting an analyte, comprising a substrate with a proximal region and a distal region; a conductive pattern formed on the substrate including at least one electrode disposed on the substrate at the proximal region of the strip, electrical strip contacts disposed on the substrate at a conductive region at the distal region of the test strip, and conductive traces electrically connecting the electrodes to at least some of the electrical strip contacts; and a reagent layer contacting at least a portion of at least one electrode, the reagent layer comprising a glucose oxidoreductase; a mediator formulation comprising at least one electroactive molecule independently selected from resazurin, resazurin salt, resorufin or resorufin salt; and at least one metal coordination complex. In some embodiments, the glucose oxidoreductase is glucose dehydrogenase. In some aspects, the present disclosure further involves a coenzyme. In some embodiments, the coenzyme is flavin adenine dinucleotide. In some embodiments, the coenzyme is nicotinamide adenine dinucleotide. In some embodiments, the at least one metal coordination complex is a ruthenium complex or an osmium complex.

In some aspects, the present disclosure provides a reagent for detecting an analyte, the reagent comprising resazurin sodium salt or resorufin sodium salt.

In some aspects, the present disclosure provides a system for measuring a body fluid constituent comprising a test strip for detecting an analyte comprising a substrate with a proximal region and a distal region; a conductive pattern formed on the substrate including at least one electrode disposed on the substrate at the proximal region of the strip, electrical strip contacts disposed on the substrate at a conductive region at the distal region of the test strip, and conductive traces electrically connecting the electrodes to at least some of the electrical strip contacts; and a reagent layer contacting at least a portion of at least one electrode, the reagent layer comprising a glucose oxidoreductase; a mediator formulation comprising at least one electroactive molecule independently selected from resazurin, resazurin salt, resorufin or resorufin salt; and at least one metal coordination complex; and a diagnostic meter configured to receive the test strip and to electrically connect to the electrical strip contacts. In some embodiments, the glucose oxidoreductase is glucose dehydrogenase. In some aspects, the present disclosure further involves a coenzyme. In some embodiments, the coenzyme is flavin adenine dinucleotide. In some embodiments, the coenzyme is nicotinamide adenine dinucleotide. In some embodiments, the at least one metal coordination complex is a ruthenium complex or an osmium complex.

In some embodiments, the reagent layer comprises an oxidoreductase, a coenzyme, and a mediator formulation, where oxidoreductase is glucose oxidoreductase and the coenzyme is selected from a flavin nucleotide and/or a nicotinamide nucleotide. The mediator formulation comprises at least one electroactive molecule and at least one coordination complex. The at least one electroactive molecule may be independently selected from phenoxazines, or salts thereof, while the at least one coordination complex is independently selected from iron, osmium or ruthenium complexes. In an embodiment, the at least one electroactive molecule is a phenoxazine or a salt thereof and the at least one coordination complex is a ruthenium complex.

In some embodiments, the reagent layer comprises an oxidoreductase, a coenzyme, and a mediator formulation, where the oxidoreductase is glucose dehydrogenase (GDH) and the coenzyme is selected from flavin adenine dinucleotide (FAD+) and/or nicotinamide adenine dinucleotide (NAD+). The mediator formulation comprises at least one electroactive molecule and at least one coordination complex. The at least one electroactive molecule may be a phenoxazine or a salt thereof and the at least one coordination complex may be a ruthenium complex. In an embodiment, the ruthenium complex is ruthenium hexaammine.

In another embodiment, the reagent layer comprises an oxidoreductase, a coenzyme, and a mediator formulation, where the oxidoreductase is glucose dehydrogenase (GDH) and the coenzyme is selected from flavin adenine dinucleotide (FAD+) and/or nicotinamide adenine dinucleotide (NAD+). The mediator formulation comprises at least one electroactive molecule and at least one coordination complex. The at least one electroactive molecule may be a phenoxazine or a salt thereof, and the at least one coordination complex may be a ruthenium complex. In an embodiment, the at least one electroactive molecule is resazurin or a salt thereof. In an embodiment, the at least one electroactive molecule is resorufin or a salt thereof.

In another embodiment, the reagent layer comprises an oxidoreductase, a coenzyme, and a mediator formulation, where the oxidoreductase is glucose dehydrogenase (GDH) and the coenzyme is selected from flavin adenine dinucleotide (FAD+) and/or nicotinamide adenine dinucleotide (NAD+). The mediator formulation comprises at least one electroactive molecule and at least one coordination complex. The at least one electroactive molecule may be independently selected from resazurin or a salt thereof or resorufin or a salt thereof, and the at least one coordination complex may be a ruthenium complex. In an embodiment, the at least one coordination complex is ruthenium hexaammine.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A reagent for detecting an analyte, comprising:
an enzyme comprising glucose oxidoreductase;
at least one electroactive molecule selected from resazurin, resazurin salt, resorufin or resorufin salt; and
at least one metal coordination complex.

2. The reagent according to claim 1, wherein the glucose oxidoreductase is glucose dehydrogenase.

3. The reagent according to claim 1, wherein the at least one metal coordination complex comprises a ruthenium complex or an osmium complex.

4. The reagent according to claim 1, wherein the at least one electroactive molecule is provided in concentration of between 0.01 mM and about 20 mM.

5. The reagent according to claim 1, wherein the at least one electroactive molecule comprises resazurin or resazurin salt is provided in concentration of between 1 mM and about 3 mM.

6. The reagent according to claim 1, wherein the at least one electroactive molecule comprises resorufin or resorufin salt is provided in concentration of between 1 mM and about 4 mM.

7. The reagent according to claim 1, further comprising a coenzyme comprising flavin adenine dinucleotide or nicotinamide adenine dinucleotide.

8. A test strip for detecting an analyte, comprising:
a substrate with a proximal region and a distal region;
a conductive pattern formed on the substrate including at least one electrode disposed on the substrate at the proximal region of the substrate, electrical strip contacts disposed on the substrate at a conductive region at the distal region of the substrate, and conductive traces electrically connecting the at least one electrode to at least some of the electrical strip contacts; and
a reagent layer contacting at least a portion of at least one electrode, the reagent layer comprising:
a glucose oxidoreductase;
a mediator formulation comprising at least one electroactive molecule independently selected from resazurin, resazurin salt, resorufin or resorufin salt; and
at least one metal coordination complex.

9. The test strip according to claim 8, wherein the glucose oxidoreductase is glucose dehydrogenase.

10. The test strip according to claim 8, wherein the at least one metal coordination complex comprises a ruthenium complex or an osmium complex.

11. The test strip according to claim 8, wherein the at least one electroactive molecule is provided in concentration of between 0.01 mM and about 20 mM.

12. The test strip according to claim 8, wherein the at least one electroactive molecule comprises resazurin or resazurin salt is provided in concentration of between 1 mM and about 3 mM.

13. The test strip according to claim 8, wherein the at least one electroactive molecule comprises resorufin or resorufin salt is provided in concentration of between 1 mM and about 4 mM.

14. The test strip according to claim 8, further comprising a coenzyme comprising flavin adenine dinucleotide or nicotinamide adenine dinucleotide.

15. A system for measuring a body fluid constituent comprising:
a test strip for detecting an analyte comprising:
a substrate with a proximal region and a distal region;
a conductive pattern formed on the substrate including at least one electrode disposed on the substrate at the proximal region of the substrate, electrical strip contacts disposed on the substrate at a conductive region at the distal region of the substrate, and conductive traces electrically connecting the at least one electrode to at least some of the electrical strip contacts; and
a reagent layer contacting at least a portion of at least one electrode, the reagent layer comprising:
a glucose oxidoreductase;
a mediator formulation comprising at least one electroactive molecule independently selected from resazurin, resazurin salt, resorufin or resorufin salt; and
at least one metal coordination complex; and
a diagnostic meter configured to receive the test strip and to electrically connect to the electrical strip contacts.

16. The system according to claim 15, wherein the glucose oxidoreductase is glucose dehydrogenase.

17. The system according to claim 15, wherein the at least one metal coordination complex comprises a ruthenium complex or an osmium complex.

18. The system according to claim 15, wherein the at least one electroactive molecule is provided in concentration of between 0.01 mM and about 20 mM.

19. The system according to claim 15, wherein the at least one electroactive molecule comprises resazurin or resazurin salt is provided in concentration of between 1 mM and about 3 mM.

20. The system according to claim 15, wherein the at least one electroactive molecule comprises resorufin or resorufin salt is provided in concentration of between 1 mM and about 4 mM.
